(19) United States
(12) Patent Application Publication
(10) Pub. No.: US 2022/0114672 A1
(43) Pub. Date: Apr. 14, 2022

(54) SYSTEMS AND METHODS FOR PROCESSING PAPER BILLS

(71) Applicant: Mitchell International, Inc., San Diego, CA (US)

(72) Inventor: Michael Cwynar, San Diego, CA (US)

(73) Assignee: Mitchell International, Inc., San Diego, CA (US)

(21) Appl. No.: 17/069,639

(22) Filed: Oct. 13, 2020

(51) Int. Cl. G06Q 40/08; G06Q 10/10; G06Q 30/04; G06Q 30/00; G06Q 50/26; G16H 40/20; G06K 7/14; G06K 19/06; G06K 15/02; G06F 40/174

(52) U.S. Cl. CPC G06Q 40/08; G06Q 10/10; G06Q 30/04; G06Q 30/0185; G06Q 50/26; G06Q 2220/00; G06K 7/1417; G06K 19/06037; G06K 15/1852; G06F 40/174; G16H 40/20

(57) ABSTRACT

Systems and methods for processing paper bills are provided. In general, one aspect disclosed features a computer-implemented method, comprising: at a first computing device: receiving first data to be printed as text on a first form, encoding the received first data as a two-dimensional machine-readable code, and causing the first form to be printed on paper, including the text and the two-dimensional machine-readable code; and at a second computing device: receiving second data representing a scan of the printed two-dimensional machine-readable code, obtaining the first data from the received second data, and populating a second form according to the obtained first data.

### DESCRIPTION OF RELATED ART

The disclosed technology relates generally to systems for processing paper bills, and more particularly, some embodiments relate to a systems and methods for implementing the same.

### SUMMARY

In general, one aspect disclosed features a system, comprising: a hardware processor; and a non-transitory machine-readable storage medium encoded with instructions executable by the hardware processor to perform a method comprising: at a first computing device: receiving first data to be printed as text on a first form, encoding the received first data as a two-dimensional machine-readable code, and causing the first form to be printed on paper, including the text and the two-dimensional machine-readable code; and at a second computing device: receiving second data representing a scan of the printed two-dimensional machine-readable code, obtaining the first data from the received second data, and populating a second form according to the obtained first data.

Embodiments of the system may include one or more of the following features. In some embodiments, the first form represents an insurance bill; and the second form represents an adjusted insurance bill. In some embodiments, the method further comprises adjusting a portion of the obtained first data according to one or more regulations, and populating the second form according to the adjusted first data. In some embodiments, the two-dimensional machine-readable code is a Quick Response (QR) code. In some embodiments, the method further comprises at the second computing device, causing the second form to be printed on paper. In some embodiments, encoding the received first data as a two-dimensional machine-readable code comprises: encrypting the received first data; and encoding the encrypted first data as the two-dimensional machine-readable code. In some embodiments, obtaining the first data from the received second data comprises: decrypting the second data.

In general, one aspect disclosed features a non-transitory machine-readable storage medium encoded with instructions executable by a hardware processor of a computing component, the machine-readable storage medium comprising instructions to cause the hardware processor to perform a method comprising: at a first computing device: receiving first data to be printed as text on a first form, encoding the received first data as a two-dimensional machine-readable code, and causing the first form to be printed on paper, including the text and the two-dimensional machine-readable code; and at a second computing device: receiving second data representing a scan of the printed two-dimensional machine-readable code, obtaining the first data from the received second data, and populating a second form according to the obtained first data.

Embodiments of the non-transitory machine-readable storage medium may include one or more of the following features. In some embodiments, the first form represents an insurance bill; and the second form represents an adjusted insurance bill. In some embodiments, the method further comprises adjusting a portion of the obtained first data according to one or more regulations, and populating the second form according to the adjusted first data. In some embodiments, the two-dimensional machine-readable code is a Quick Response (QR) code. In some embodiments, the method further comprises at the second computing device, causing the second form to be printed on paper. In some embodiments, encoding the received first data as a two-dimensional machine-readable code comprises: encrypting the received first data; and encoding the encrypted first data as the two-dimensional machine-readable code. In some embodiments, obtaining the first data from the received second data comprises: decrypting the second data.

In general, one aspect disclosed features a computer-implemented method, comprising: at a first computing device: receiving first data to be printed as text on a first form, encoding the received first data as a two-dimensional machine-readable code, and causing the first form to be printed on paper, including the text and the two-dimensional machine-readable code; and at a second computing device: receiving second data representing a scan of the printed two-dimensional machine-readable code, obtaining the first data from the received second data, and populating a second form according to the obtained first data.

Embodiments of the method may include one or more of the following features. In some embodiments, the first form represents an insurance bill; and the second form represents an adjusted insurance bill. Some embodiments comprise adjusting a portion of the obtained first data according to one or more regulations, and populating the second form according to the adjusted first data. In some embodiments, the two-dimensional machine-readable code is a Quick Response (QR) code. Some embodiments comprise at the second computing device, causing the second form to be printed on paper. In some embodiments, encoding the received first data as a two-dimensional machine-readable code comprises: encrypting the received first data, and encoding the encrypted first data as the two-dimensional machine-readable code; and obtaining the first data from the received second data comprises: decrypting the second data.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 illustrates a conventional system for processing paper insurance bills.

FIG. 2 illustrates a paper bill processing system according to some embodiments of the disclosed technology.

FIGS. 3A-C illustrate a process for paper bill processing according to some embodiments of the disclosed technology.

FIG. 4 illustrates an example insurance bill with QR code according to some embodiments of the disclosed technologies.

FIG. 5 illustrates another example insurance bill with QR code according to some embodiments of the disclosed technologies.

FIG. 6 is an example computing component that may be used to implement various features of embodiments described in the present disclosure.

The figures are not exhaustive and do not limit the present disclosure to the precise form disclosed.

### DETAILED DESCRIPTION

Insurance carriers or payers submit electronic medical bills for review to determine whether the bill should be disqualified or rejected or whether the associated fee is accurate or should be adjusted, for example according to factors such as jurisdictional regulations, proprietary edits, industry standard practices, correct coding, provider fraud, duplicate checks, billing errors, other payment calculations, and the like. The electronic medical bills may be workers compensation or auto casualty medical bills, for example, although other types of bills may be submitted for an automated review as well.

The vast majority of such bills are submitted in paper form, and in very large amounts. Electronic billing regulations exist across many state jurisdictions. However, those regulations mandate only that the payer be able to accept electronic bills. There is no mandate that requires providers/doctors/billing agencies to submit bills electronically. Industry statistics exist that show only 10-20% of the bills processed for workers compensation and auto casualty are submitted electronically. With nearly 100 million bills submitted annually, a large amount of human capacity is required to convert those bills from paper to an electronic format for final processing and payment.

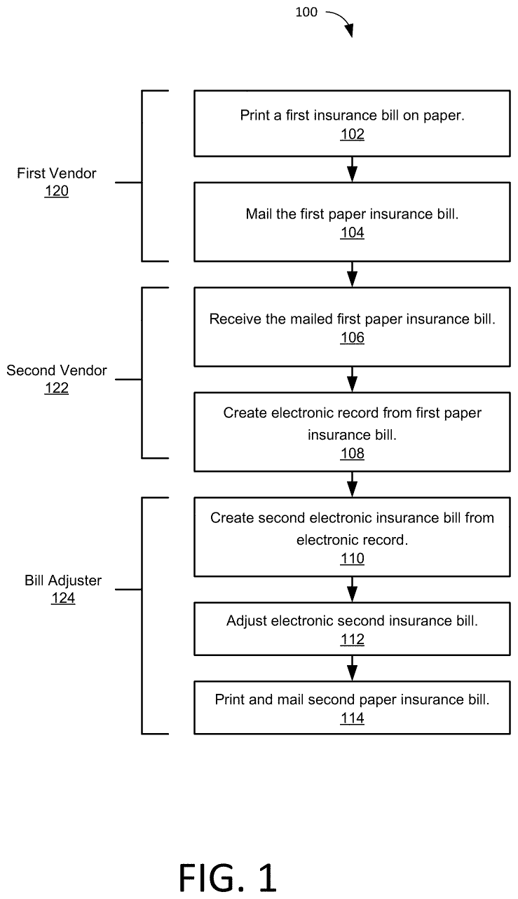

FIG. 1 illustrates a conventional system 100 for processing paper insurance bills. Referring to FIG. 1, the process 100 begins with the printing a first insurance bill on paper, at 102, and mailing the first paper insurance bill, at 104. The first paper insurance bill may be printed and mailed by a first vendor 120, for example using an Electronic Medical Records (EMR) system or the like.

A second vendor 122 receives the first paper insurance bill, at 106, and creates an electronic record from the first paper insurance bill, at 108. Various techniques are employed in the process of creating an electronic record from a paper insurance bill. For example, the second vendor may scan the paper bill, perform Optical Character Recognition (OCR) on the scanned paper bill, or simply manually enter the data from the paper bill into an electronic form. However, these techniques are not only costly and time consuming, but also prone to the introduction of errors in the data. Scans and OCR are unreliable, especially given the poor quality of many bills, which may have been copied, faxed, scanned, and the like. These processes may be used anyway, but require human oversight, while manual data entry is subject to human error. But these are the techniques currently employed.

Once the electronic record of the paper bill is created, a second electronic insurance bill is created, at 110, for example by a bill adjuster 124. Being in electronic form, the bill is easy to share for collaboration. For example, the second electronic insurance bill may be adjusted, at 112, for example to reduce prices according to price caps, deny payment of certain lines, and the like. Once the second electronic insurance bill, it is generally printed and mailed to the customer, at 114.

Embodiments of the disclosed technology provide elements of a paper bill processing system that addresses the above-described problems. When the bill is initially printed, a QR code is also printed on the bill. The QR code includes the billing information used to create the bill. Then, after the bill is mailed and received, the billing information may be quickly and accurately recovered by scanning the QR code. This technique ensures the integrity of the billing data, while eliminating the current error-prone processes for reproducing the billing information from the paper bill. Furthermore, this technique does not require the provider to change its billing processes, which providers are quite reluctant to do.

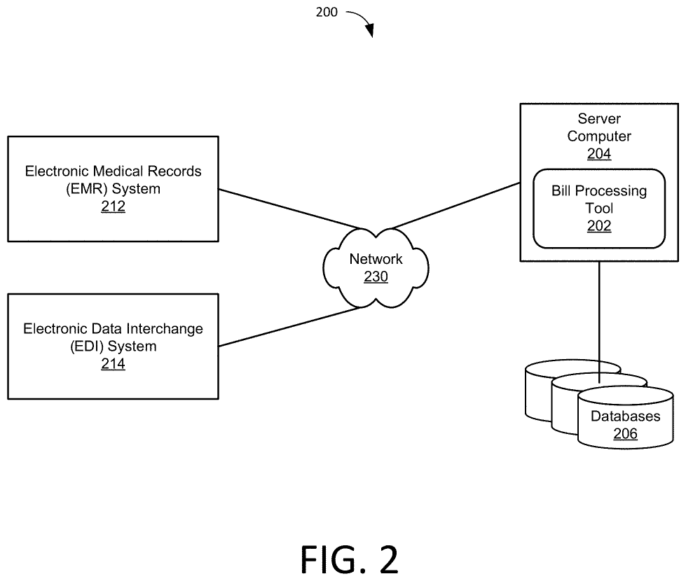

FIG. 2 illustrates a paper bill processing system 200 according to some embodiments of the disclosed technology. Referring to FIG. 2, the system 200 includes a bill processing tool 202, which may be implemented as one or more software packages executing on one or more server computers 204. The system may include one or more databases 206, which may store insurance bills, bill processing rules, and the like. The tool 202 may access the databases 206 over either a public or private network.

Multiple other systems may be involved in paper bill processing. For example, an Electronic Medical Records (EMR) System 212 may produce and mail the initial paper bills. An Electronic Data Interchange (EDI) System 214 may receive the mailed paper bills, generate electronic records from the received paper bills, and provide the electronic records to the Bill Processing Tool 202. The Bill Processing Tool 202, EMR System 212, and EDI System 214 may communicate over a network 230.

In this description, various embodiments are disclosed for processing paper medical insurance bills. However, embodiments of the disclosed technology apply to processing other paper insurance bills, for example such as auto insurance bills and the like. Furthermore, embodiments of the disclosed technology apply to processing other paper bills, other paper documents, paper forms, and the like. These and other applications will be apparent to one skilled in the relevant art after reading this description.

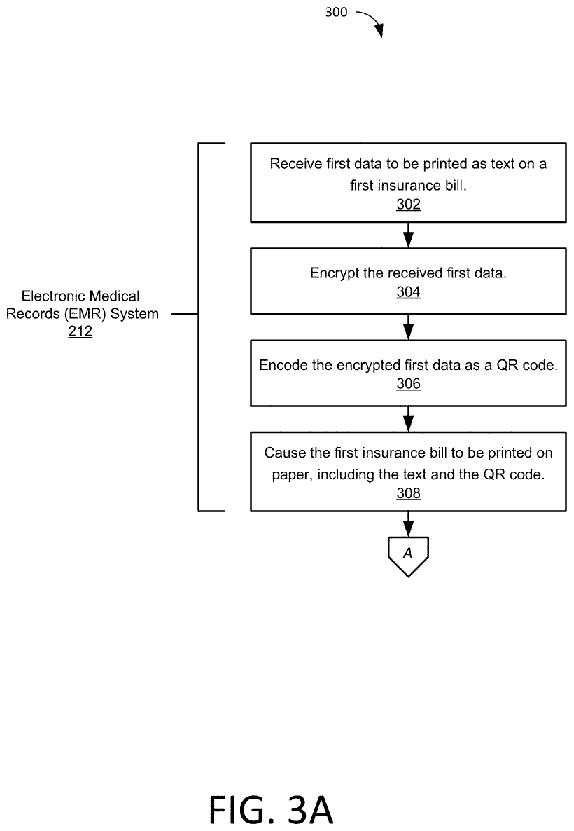

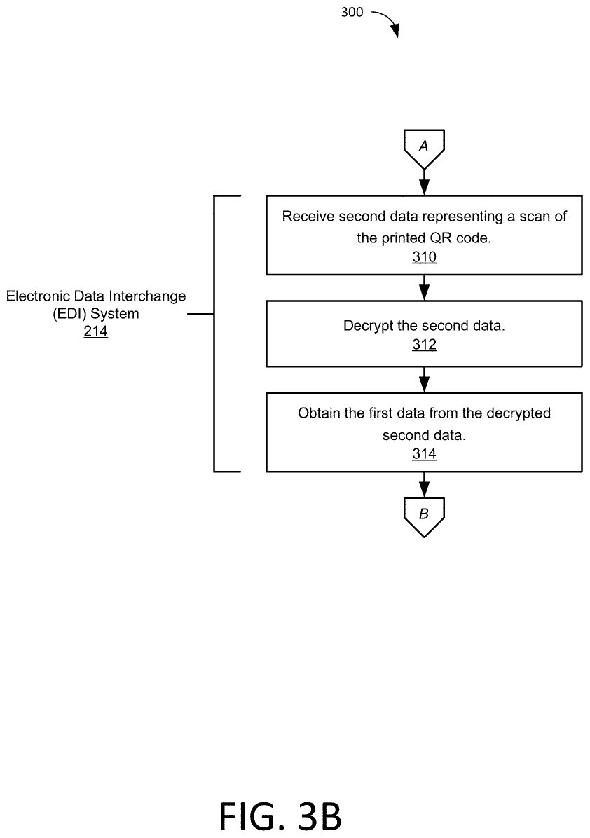

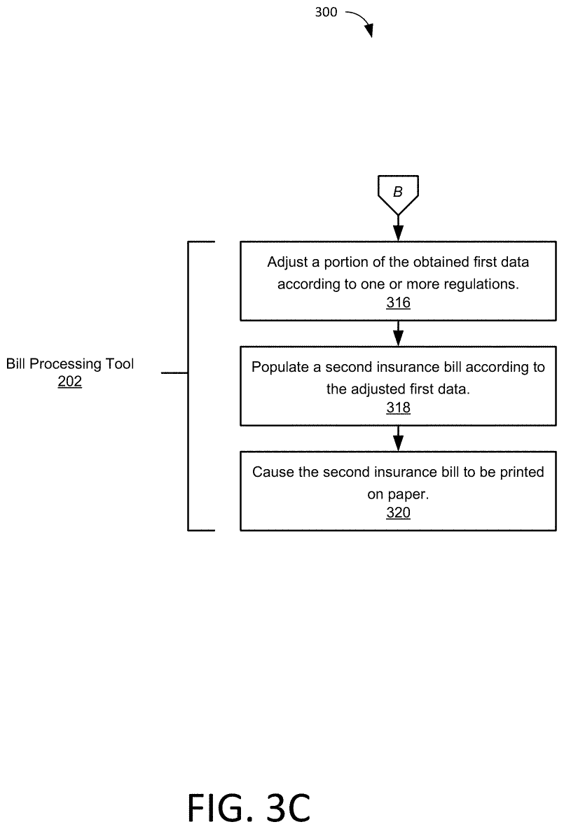

FIGS. 3A-C illustrate a process 300 for paper bill processing according to some embodiments of the disclosed technology. The process 300 may be performed, for example, by the paper bill processing system 200 of FIG. 2. The steps 302-308 of the process 300 may be performed by the EMR System 212 of FIG. 2.

Referring now to FIG. 3A, the process 300 may begin with receiving first data to be printed as text on a first insurance bill, at 302. The first data may include any data that may be used to populate an insurance bill. For example, in the case of a medical insurance bill, the information may describe the insured, medical procedures and other medical items, prices, and the like.

For increased security and privacy, in some embodiments, the process 300 may include encrypting the first data, at 304. Any encryption techniques may be used. The process 300 may include encoding the first data and/or the encrypted first data in a QR code, at 306. But while the disclosed embodiments employ QR codes, other embodiments may employ other two-dimensional machine-readable codes, as will be apparent to one skilled in the relevant art after reading this description. After generating the QR code, the process 300 may include causing the first insurance bill to be printed on paper, including the text and the QR code, at 308.

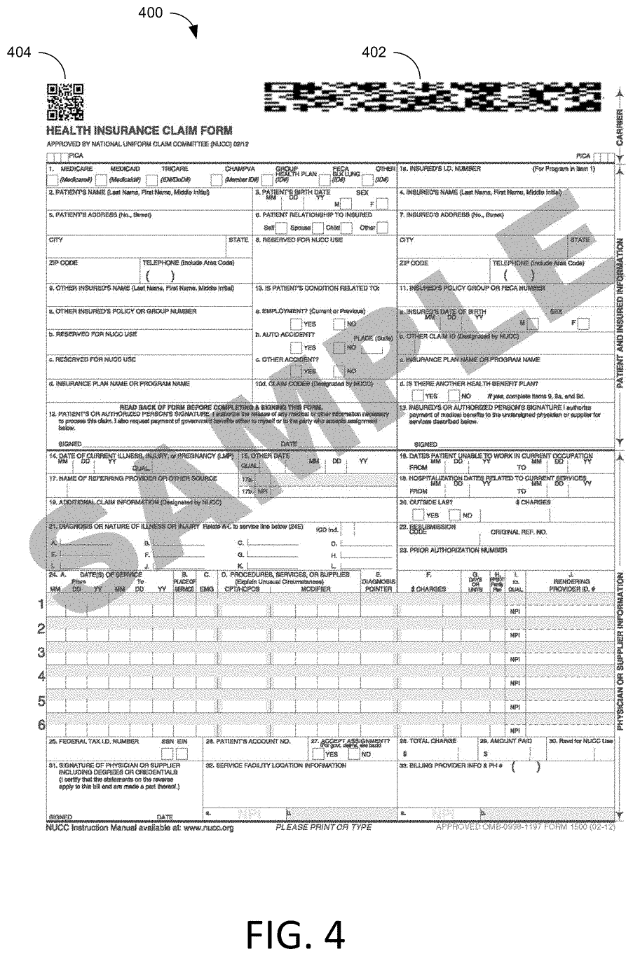

FIG. 4 illustrates an example insurance bill 400 with QR code 402 according to some embodiments of the disclosed technologies. The format and contents of medical insurance bills are often tightly controlled by applicable legal rules and regulations. In the example of FIG. 4, the insurance bill 400 is a CMS-1500 Health Insurance Claim Form approved by the National Uniform Claim Committee (NUCC). The insurance bill 400 includes an example QR code 402 that represents data entered on the insurance bill 400. Note in this example, the text fields of the insurance bill 400 are blank, but in practice the insurance bill 400 would be populated with text. The CMS-1500 Health Insurance Claim Form includes another QR code 404 as part of the form. The QR code 404 when scanned takes the user to the NUCC landing page for the CMS-1500 form. This QR code 404 should not be confused with the QR code 402 generated according to the disclosed technologies.

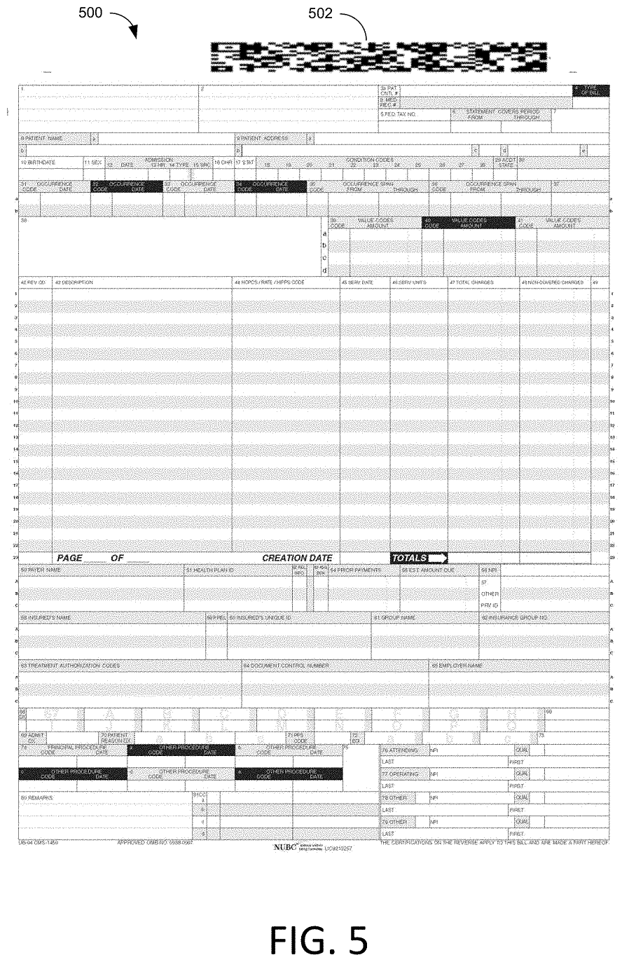

FIG. 5 illustrates another example insurance bill 500 with QR code 502 according to some embodiments of the disclosed technologies. In the example of FIG. 5, the insurance bill 500 is a UB-04 CMS-1450 Health Insurance Claim Form licensed from the by the National Uniform Billing Committee (NUBC). The insurance bill 500 includes an example QR code 502 that represents data entered on the insurance bill 500. Note in this example, the text fields of the insurance bill 500 are blank, but in practice the insurance bill 500 would be populated with text.

The forms in FIGS. 4 and 5 are federally regulated. Therefore, it is not permissible to remove any part of the forms. However, it is permissible to add information to the forms. By adding the disclosed QR codes to the forms, the disclosed technology is compliant with federal regulations.

After printing, the paper insurance bill may be physically transmitted to a processor, where paper insurance bill may be scanned. Referring to FIG. 3B, steps 310-314 of the process 300 may be performed by the EDI System 214 of FIG. 2. The process 300 may include receiving second data representing a scan of the printed QR code, at 310. In some embodiments, the received second data may be encrypted. In some embodiments, the process 300 may include decrypting the received second data, at 312. In some embodiments, the second data and the first data may be the same. In other embodiments, the second data may represent the first data. In some embodiments, the process 300 may include obtaining the first data from the decrypted second data, at 314. The first data may take the form of an electronic record representing the insurance bill.

The electronic record may be transmitted electronically to a bill processor. Referring to FIG. 3C, steps 316-320 of the process 300 may be performed by the Bill Processing Tool 202 of FIG. 2. The process 300 may include adjusting a portion of the obtained first data according to one or more rules, regulations, and the like, at 316. For example, the electronic record representing the insurance bill may be generally processed by a rule engine that applies rules that are highly dependent on the first data, for example according to factors such as pricing, pre-pricing, various data checks, scrubbing, editing, warning, pricing and other adjustment rules affecting the final outcome of the bill, and the like. This adjustment process may be automated, manual, or a combination thereof.

After adjusting the first data, the process 300 may include populating a second insurance bill according to the adjusted first data, at 318. For example, the process 300 may use the first data to populate fields of an electronic form representing the second insurance bill. After the second insurance bill is populated, the process 300 may include causing the second insurance bill to be printed on paper, at 320. After printing, the second paper insurance bill may be physically transmitted to the customer.

The disclosed technology provides multiple advantages over conventional paper bill processing techniques. One advantage, which benefits the provider/doctor/billing agency, is that the contents of the printed bill are stored in a machine-readable format that does not require additional work on the agency's part. This advantage is a major differentiator to existing electronic billing solutions because the disclosed technology requires no changes in workflow or signing up to use a new external solution. A second benefit is to entities that handle the paper bills. By storing the contents of the bill in a multidimensional machine-readable format, these entities will own the solution for quickly reading the bill contents and properly formatting the bill contents for electronic storage and processing.

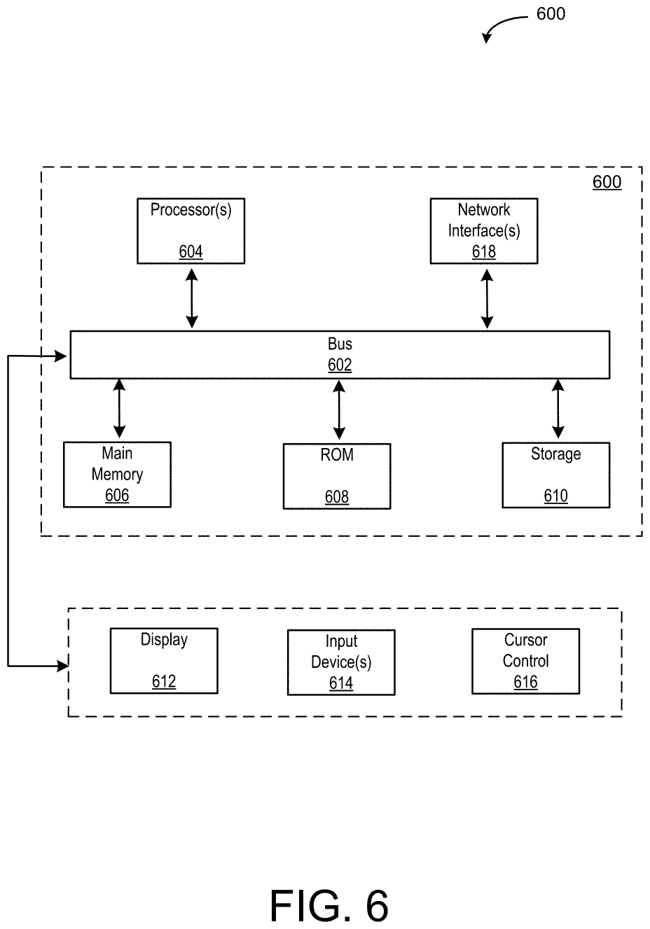

FIG. 6 depicts a block diagram of an example computer system 600 in which embodiments described herein may be implemented. The computer system 600 includes a bus 602 or other communication mechanism for communicating information, one or more hardware processors 604 coupled with bus 602 for processing information. Hardware processor(s) 604 may be, for example, one or more general purpose microprocessors.

The computer system 600 also includes a main memory 606, such as a random access memory (RAM), cache and/or other dynamic storage devices, coupled to bus 602 for storing information and instructions to be executed by processor 604. Main memory 606 also may be used for storing temporary variables or other intermediate information during execution of instructions to be executed by processor 604. Such instructions, when stored in storage media accessible to processor 604, render computer system 600 into a special-purpose machine that is customized to perform the operations specified in the instructions.

The computer system 600 further includes a read only memory (ROM) 608 or other static storage device coupled to bus 602 for storing static information and instructions for processor 604. A storage device 610, such as a magnetic disk, optical disk, or USB thumb drive (Flash drive), etc., is provided and coupled to bus 602 for storing information and instructions.

The computer system 600 may be coupled via bus 602 to a display 612, such as a liquid crystal display (LCD) (or touch screen), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. In some embodiments, the same direction information and command selections as cursor control may be implemented via receiving touches on a touch screen without a cursor.

The computing system 600 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component,” “engine,” “system,” “database,” data store,” and the like, as used herein, can refer to logic embodied in hardware or firmware, or to a collection of software instructions, possibly having entry and exit points, written in a programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor(s) 604 executing one or more sequences of one or more instructions contained in main memory 606. Such instructions may be read into main memory 606 from another storage medium, such as storage device 610. Execution of the sequences of instructions contained in main memory 606 causes processor(s) 604 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 610. Volatile media includes dynamic memory, such as main memory 606. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 602. Transmission media can also take the form of acoustic or light waves, such as those generated during radio-wave and infra-red data communications.

The computer system 600 also includes a communication interface 618 coupled to bus 602. Network interface 618 provides a two-way data communication coupling to one or more network links that are connected to one or more local networks. For example, communication interface 618 may be an integrated services digital network (ISDN) card, cable modem, satellite modem, or a modem to provide a data communication connection to a corresponding type of telephone line. As another example, network interface 618 may be a local area network (LAN) card to provide a data communication connection to a compatible LAN (or a WAN component to communicate with a WAN). Wireless links may also be implemented. In any such implementation, network interface 618 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet.” Local network and Internet both use electrical, electromagnetic or optical signals that carry digital data streams. The signals through the various networks and the signals on network link and through communication interface 618, which carry the digital data to and from computer system 600, are example forms of transmission media.

The computer system 600 can send messages and receive data, including program code, through the network(s), network link and communication interface 618. In the Internet example, a server might transmit a requested code for an application program through the Internet, the ISP, the local network and the communication interface 618.

The received code may be executed by processor 604 as it is received, and/or stored in storage device 610, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, a circuit might be implemented utilizing any form of hardware, or a combination of hardware and software. For example, one or more processors, controllers, ASICs, PLAs, PALs, CPLDs, FPGAs, logical components, software routines or other mechanisms might be implemented to make up a circuit. In implementation, the various circuits described herein might be implemented as discrete circuits or the functions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 600.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A system, comprising:
a hardware processor; and
a non-transitory machine-readable storage medium encoded with instructions executable by the hardware processor to perform a method comprising:
at a first computing device:
receiving first data to be printed as text on a first form,
encoding the received first data as a two-dimensional machine-readable code, and
causing the first form to be printed on paper, including the text and the two-dimensional machine-readable code; and
at a second computing device:
receiving second data representing a scan of the printed two-dimensional machine-readable code,
obtaining the first data from the received second data, and
populating a second form according to the obtained first data.

2. The system of claim 1, wherein:
the first form represents an insurance bill; and
the second form represents an adjusted insurance bill.

3. The system of claim 2, the method further comprising:
adjusting a portion of the obtained first data according to one or more regulations, and
populating the second form according to the adjusted first data.

4. The system of claim 1, wherein:
the two-dimensional machine-readable code is a Quick Response (QR) code.

5. The system of claim 1, the method further comprising:
at the second computing device, causing the second form to be printed on paper.

6. The system of claim 1, wherein encoding the received first data as a two-dimensional machine-readable code comprises:
encrypting the received first data; and
encoding the encrypted first data as the two-dimensional machine-readable code.

7. The system of claim 6, wherein obtaining the first data from the received second data comprises:
decrypting the second data.

8. A non-transitory machine-readable storage medium encoded with instructions executable by a hardware processor of a computing component, the machine-readable storage medium comprising instructions to cause the hardware processor to perform a method comprising:
at a first computing device:
receiving first data to be printed as text on a first form,
encoding the received first data as a two-dimensional machine-readable code, and
causing the first form to be printed on paper, including the text and the two-dimensional machine-readable code; and
at a second computing device:
receiving second data representing a scan of the printed two-dimensional machine-readable code,
obtaining the first data from the received second data, and
populating a second form according to the obtained first data.

9. The non-transitory machine-readable storage medium of claim 8, wherein:
the first form represents an insurance bill; and
the second form represents an adjusted insurance bill.

10. The non-transitory machine-readable storage medium of claim 9, the method further comprising:
adjusting a portion of the obtained first data according to one or more regulations, and
populating the second form according to the adjusted first data.

11. The non-transitory machine-readable storage medium of claim 8, wherein:
the two-dimensional machine-readable code is a Quick Response (QR) code.

12. The non-transitory machine-readable storage medium of claim 8, the method further comprising:
at the second computing device, causing the second form to be printed on paper.

13. The non-transitory machine-readable storage medium of claim 8, wherein encoding the received first data as a two-dimensional machine-readable code comprises:
encrypting the received first data; and
encoding the encrypted first data as the two-dimensional machine-readable code.

14. The non-transitory machine-readable storage medium of claim 13, wherein obtaining the first data from the received second data comprises:
decrypting the second data.

15. A computer-implemented method, comprising:
at a first computing device:
receiving first data to be printed as text on a first form,
encoding the received first data as a two-dimensional machine-readable code, and
causing the first form to be printed on paper, including the text and the two-dimensional machine-readable code; and
at a second computing device:
receiving second data representing a scan of the printed two-dimensional machine-readable code,
obtaining the first data from the received second data, and
populating a second form according to the obtained first data.

16. The computer-implemented method of claim 15, wherein:
the first form represents an insurance bill; and
the second form represents an adjusted insurance bill.

17. The computer-implemented method of claim 16, further comprising:
adjusting a portion of the obtained first data according to one or more regulations, and
populating the second form according to the adjusted first data.

18. The computer-implemented method of claim 15, wherein:
the two-dimensional machine-readable code is a Quick Response (QR) code.

19. The computer-implemented method of claim 15, further comprising:
at the second computing device, causing the second form to be printed on paper.

20. The computer-implemented method of claim 15, wherein:
encoding the received first data as a two-dimensional machine-readable code comprises:
encrypting the received first data, and
encoding the encrypted first data as the two-dimensional machine-readable code; and
obtaining the first data from the received second data comprises:
decrypting the second data.
